(19) United States
(12) Patent Application Publication
(10) Pub. No.: US 2003/0206946 A1
(43) Pub. Date: Nov. 6, 2003

(54) Methods for therapy of connective tissue disease

(76) Inventor: Yih-Lin Chung, Taipei (TW)

Correspondence Address: FISH & RICHARDSON PC, 225 FRANKLIN ST, BOSTON, MA, 02110, US

(21) Appl. No.: 10/132,999

(22) Filed: Apr. 26, 2002

(51) Int. Cl. A61K 38/16; A61K 31/445; A61K 9/127; A61K 31/19; A61K 31/165

(52) U.S. Cl. 424/450; 514/2; 514/557; 514/570; 514/617

(57) ABSTRACT

Compounds of a histone deacetylase inhibitor. The compounds are capable of simultaneously modulating cell cycle-related gene expression and inflammatory cytokine production, and are useful as anti-inflammatory agents in connective tissue disease resulting from altered patterns of immunoregulation. The present invention provides methods of treating, preventing or ameliorating connective tissue diseases, which share common features including inflammation of skin, joints and soft tissues as well as altered patterns of immunoregulation, by administering a therapeutically effective amount of a histone deacetylase inhibitor.

### BACKGROUND OF THE INVENTION

1. Field of the invention

The present invention relates to a pharmaceutical composition and method for the treatment of connective tissue diseases. More particularly, the present invention relates to a pharmaceutical composition and method for the treatment of inflammation of skin, joints and soft tissues due to altered patterns of immunoregulation such as rheumatoid arthritis, systemic lupus erythematosus, progressive systemic sclerosis, sjogren's syndrome, dermatomyositis and mixed connective tissue disease.

2. Description of the Related Art

The disease group of connective tissue disease in general is an autoimmune disorder in which genetic factors appear to play a role although the pathogenesis remains incompletely understood. Connective tissue diseases have in common widespread immunologic and inflammatory alterations of connective tissue. Common findings include arthritis or synovitis, pleuritis, myocarditis, endocarditis, pericarditis, peritonitis, vasculitis, myositis, dermatitis, nephritis, and alterations of connective tissues. Connective tissue disease involves virtually any organ system. Course of disease is one of periods of exacerbation and remission.

Among the group of connective tissue diseases, rheumatoid arthritis is the most common form of inflammatory arthritis. The main pathology of the affected joints consists of synovial hyperplasia and pannus formation, bone and cartilage destruction, subintimal infiltration of T and B lymphocytes, and production and/or induction of proinflammatory mediators from macrophages and fibroblasts. The proliferation of the mesenchymal and fibroblast-like synovial cells responding to the inflammatory cytokine milieu and transforming into tumor-like qualities leads to irreversible cartilage and bone destruction.

To date, no truly effective or curative therapeutic drug has resulted in unsatisfactory outcomes and an increased awareness of the cost, lost productivity, morbidity, and decreased life expectancy, which are the consequences of progressively recurrent disease. There is a need for effective therapies to prevent skin, joint or soft tissue destruction and maintain functional status.

### SUMMARY OF THE INVENTION

According to the present invention it was surprisingly found that histone deacetylase inhibitors and in particular of trichostatin A and phenylbutyrate strongly inhibit main features of connective tissue disease of inflammation of skin, joints, and soft tissues, which results in prevention of skin ulcers, joint destruction, and soft tissue necrosis and swelling. That simultaneously the synovial tissue hyperplasia is blocked, the bone and cartilage are preserved, the lymphocyte infiltration is decreased, and the proinflammatory mediator is suppressed indicates that trichostatin A and phenylbutyrate are potent agents for the treatment of connective tissue disease.

The present invention is directed to the use of a histone deacetylase inhibitor and a pharmaceutically acceptable carrier or a pharmaceutically acceptable salt thereof for the preparation of a pharmaceutical composition for the treatment of connective tissue disease.

Histone deacetylase inhibitors are substances causing an inhibition of the activity of histone deacetylases, resulting in hyperacetylation. Currently compounds shown to inhibit the activity of histone deacetylase fall into six structurally diverse classes, comprising: phenylbutyrate of the short chain fatty acid class, depudecin of the epoxide class, trapoxin A of the cyclic tetrapeptide class containing a 2-amino-8-oxo-9, 10-epoxy-decanoyl moiety, depsipeptide of the cyclic tetrapeptide class lacking a 2-amino-8-oxo-9, 10-epoxy-decanoyl moiety, trichostatin A of the hydroxamic acid class, and the benzamide class.

Phenylbutyrate inhibits histone deacetylases by a noncompetitive mechanism at millimolar concentrations. Trichostatin A is a specific inhibitor of histone deacetylase, and effective in the submicromolar range.

### BRIEF DESCRIPTION OF THE DRAWINGS

The present invention will be more fully understood and further advantages will become apparent when reference is made to the following description of the invention and the accompanying drawings in which:

FIGS. 1A and 1B are western blot results showing the accumulation of acetylated histones induced by phenylbutyrate (2 mM) (A) and trichostatin A (200 nM) (B) at different time interval in Example 1.

FIGS. 2A-2C are photographs of immunofluorescence with anti-acetylated H3 antibody showing the accumulation of acetylated histones induced by phenylbutyrate (B) and trichostatin A (C) and a negative control (A) in synovial fibroblasts in Example 1.

FIGS. 3A and 3B are diagrams showing phenylbutyrate (A) and trichostatin A (B) induces a dose- and time-dependent growth arrest in synovial fibroblast from adjuvant arthritis. The results were expressed as percentage value relative to the control culture (no drug addition). Data presented are the mean±SE of triplicate determination from two experiments.

FIGS. 4A and 4B are western blot results showing the upregulation of p16 and p21 induced by phenylbutyrate (2 mM) (A) and trichostatin A (200 nM) (B) treatment for 12 hr in synovial fibroblasts from adjuvant arthritis in Example 1. Lane 1: control (no any treatment); lane 2: phenylbutyrate-treated; lane 3: trichostatin A-treated.

FIGS. 5A-5F are photographs of immunofluorescence with anti-acetylated H3 antibody showing that treatment with 10% of phenylbutyrate cream or 1% trichostatin A ointment induces accumulation of acetylated histones in vivo in Example 3. FIGS. 5A and 5B are a control group; FIGS. 5C and 5D are a phenylbutyrate-treated group; FIGS. 5E and 5F are a trichostatin A-treated group. 5A, 5C and 5E are at Day 1; 5B, 5D and 5F are at Day 15.

FIGS. 6A-6P are photographs showing that phenylbutyrate and trichostatin A have similar effects on suppressing the connective tissue disease in Example 3. FIGS. 6A, 6C, 6E, 6G, 6I, 6K, 6M and 6O are vehicle group; FIGS. 6B, 6D, 6F, 6H, 6J, 6L, 6N and 6P are treated group. FIGS. 6A and 6B are lateral view of ankle and plantar joints; FIGS. 6C and 6D are back views of paw joints. FIGS. 6E and 6F are cross-sections (sagittal cut) of paw joints. 6G and 6H are micro-sections of paw joints. 6I, 6J, 6M and 6N are histological staining results at 40× field. 6K, 6L, 6O and 6P are histological staining results at 100× field.

FIGS. 7A-7F are histological staining results showing that trichostatin A with 1% ointment is more potent than phenylbutyrate with 10% cream in Example 3. FIGS. 7A, 7C and 7E are phenylbutyrate-treated; 7B, 7D and 7F are trichostatin A treated. FIGS. 7A and 7B are 40× field; 7C and 7D are 40× field; FIGS. 7E and 7F are 200× field.

FIGS. 8A-8L are histological staining results showing the effect of phenylbutyrate inhibitors on the cell cycle regulator in the inflamed invasive synovium of connective tissue disease in Example 4. FIGS. 8A-8F are a phenylbutyrate treated group; FIGS. 8G-8L are vehicle group. FIGS. 8A, 8C, 8E, 8G, 8I and 8K are H&E stained; FIGS. 8B, 8D, 8F, 8H, 8J and 8L are p16 immunohistochemistry stained. FIGS. 8A, 8B, 8G and 8H are 40× field; FIGS. 8C, 8D, 8I, 8J are 100× field; FIGS. 8E, 8F, 8K, 8L are 200× field.

FIGS. 9A-9J are TNF-α immunohistochemistry staining results showing the effect of phenylbutyrate on the proinflammatory mediator, TNF-α, in the connective tissues of connective tissue disease in Example 5. FIGS. 9A, 9C, 9E, 9G and 9I are vehicle group; FIGS. 9B, 9D, 9F, 9H and 9J are phenylbutyrate-treated group. FIGS. 9A and 9B are joint at 40× field; FIGS. 9C and 9D are joint at 100× field. FIGS. 9E and 9F are subcutaneous tissue at 100× field; FIGS. 9G and 9H are subcutaneous at 200× field. FIGS. 9I and 9J are dermis and epidermis at 100× field.

FIGS. 10A and 10B are immunohistochemistry staining results showing the effect of trichostatin A on p16 and TNF-αexpression in the connective tissues of connective tissue disease. FIG. 10A is p16 immunohistochemistry at 200×; FIG. 10B is TNF-α immunohistochemistry at 200α.

### DETAILED DESCRIPTION OF THE INVENTION

The description in this application is in particular directed to phenylbutyrate and trichostatin A in adjuvant arthritis, a model of connective tissue disease, as non-limiting examples and is not intended to limit the scope of the invention.

Phenylbutyrate and trichostatin A or derivatives thereof are disclosed to be useful as agents for the treatment in connective tissue disease. Pharmaceutical formulations and the use of compounds of phenylbutyrate and trichostatin A are also disclosed.

Phenylbutyrate (MW 164.21), a natural nontoxic colorless tasteless aromatic fatty acid purified from mammalian urine and plasma, is Food and Drug Administration approved for children with hyperammonemia associated with inborn errors of urea synthesis and has been used for adult patients with hyperammonemia secondary to high-dose chemotherapy. It is metabolized in the liver and kidney to phenylacetate that is subsequently conjugated with glutamine to form phenylacetylglutamine; the latter serves as vehicle for waste nitrogen excretion in the urine. Phenylbutyrate has also been evaluated in the clinical trials for sickle cell anemia, beta-thalassaemia, cystic fibrosis, adrenal leukodystrophy, and both hematological and nonhematological malignancies.

Trichostatin A (MW 164.21), a hydroxamic acid, is originally isolated from Streptomyces hygroscopicus. Trichostatin A is useful as an antifungal, anticancer, and antiprotozoal agent.

In the course of experiments phenylbutyrate and trichostatin A were discovered that they as histone deacetylase inhibitors have strongly inhibitory effects on synovial hyperplasia, pannus formation, bone and cartilage destruction, skin inflammation or ulcer or fibrosis, lymphocyte infiltration, and proinflammatory mediator production in connective tissues of connective tissue disease resulting from altered patterns of immunoregulation as an autoimmune disorder.

The histone deacetylase inhibitor agents can be brought in the form of pharmaceutically acceptable salts. As such pharmaceutically acceptable salts may be used so long as they do not adversely affect the desired pharmacological effects of the compounds. The selection and production can be performed by those skilled in the art. Examples of pharmaceutically acceptable salts include alkali metal salts such as sodium salt or a potassium salt, alkaline earth metal salts such as calcium salt or a magnesium salt, salts with an organic base such as an ammonium salt, or a salt with an organic base such as a triethylamine salt or an ethanolamine salt.

The histone deacetylase inhibitor agents of the present invention may be administered orally or non-orally. In the case of oral administration, they may be administered in the form of soft and hard capsules, tablets, granules, powders, solutions, suspensions or the like. In the case of non-oral administration, they may be administered in the form of creams, ointments, gels, lotions, patches, suppositories, liposome formations, injection solution, drip infusion formulations or the like whereby continued membrane absorption can be maintained in the form of solid, viscous liquid, or suspension. The selection of the method for the preparation of these formulations and the vehicles, disintegrators or suspending agents, can be readily made by those skilled in the art. The histone deacetylase inhibitor agents of the present invention may contain a further substance having anti-inflammatory activities, in addition to trichostatin A, or phenylbutyrate, and a pharmaceutically acceptable carrier or a pharmaceutically acceptable salt thereof.

As recognized by those skilled in the art, the effective doses vary depending on route of administration, excipient usage, and the possibility of co-use with other therapeutic treatments such as the use of other anti-inflammatory agents. Effective amounts and treatment regimens for any particular subject (e.g., human, dog, or cat) will also depend upon a variety of other factors, including the activity of the specific compound employed, age, body weight, general health status, sex, diet, time of administration, rate of excretion, severity and course of the disease, and the patient's disposition to the disease, but are usually from 0.1 to 50% by weight irrespective of the manner of administration.

In order that the invention described herein may be more readily understood, the following examples are set forth. It should be understood that these examples are for illustrative purposes only and are not to be construed as limiting this invention in any manner. All references cited herein are expressly incorporated by reference in their entirety.

### EXAMPLE 1

### Histone Deacetylase Inhibitors Induce a Dose- and Time-Dependent Growth Arrest In Synovial Fibroblasts of Adjuvant Arthritis of Connective Tissue Disease

The animal model for adjuvant arthritis, a model of connective tissue disease, has been established and characterized well (Winter CA, et al., Arthritis Rheum. 1966, 9:394-404). Long Evans rats weighing 150±20 g and ICR derived male mice weighing 22±2 g provided by animal breeding center of MDS Pharma Service-Taiwan, Ltd., were used. Space allocation for 5 mice was 45×23×15 cm. The animals were housed in APEC® (Allentown Gaging, Allentown, N.J., USA) cages and maintained in a hygienic environment under controlled temperature (22-24° C.) and humidity (60-80%) with 12-hours light/dark cycles for at least one week in MDS Pharma Service-Taiwan laboratory prior to being used. Free access to standard lab chew for mice (LabDiet® 5001, USA) and tap water was granted. All aspects of this work including housing, experimentation, and disposal of animals were performed in general according to the International Guiding Principles for Biomedical Research Involving Animals (CIOMS Publication No. ISBN 9290360194, 1985).

A well-ground suspension of killed Mycobacterium tuberculosis (DIFCO, USA; 0.3 mg in 0.1 ml of light mineral oil; Complete Freund's Adjuvant, CFA) was administered into the subplantar region of the right hind paw immediately after first dosing on first day (denoted day 1). Hind paw volume was measured by Plethsmometer (Cat. No. 7150, UGO BASILE, Italy) and Water cell (25 mm diameter, Cat. No. 7157, UGO BASILE, Italy) on day 0 (before CFA treated), 1, 5, 10, and 15 after CFA of right paw (with CFA), and day 0, 14, and 18 of left paw (without CFA); rats were weighed just before first dose and 1 hour after final dose. Right hind paw volume (Acute Phase) was measured 4 hours after CFA treated (denoted as day 1) and day 5, day 10 and day 15 after CFA. The contralateral left hind paw volume (Delayed Phase) was measured at day 14 and day 18. The inflammatory activity in this model was denoted by values calculated during the Acute Phase as well as the Delayed Phase.

At day 10, synovial samples were obtained from the established adjuvant arthritis rats. The collected synovial tissues were digested with 5 mg/ml collagenase (sigma) and 1.5 mg/ml DNase (sigma), and were passed through a wire mesh to prepare isolated cells. Cells (0.5×10⁶) were placed in 10-cm dishes with 10 ml RPMI 1640 medium (Gibco) supplemented with 10% fetal calf serum, 2 mM L-glutamine, 50 U/ml penicillin and 50 mg/ml streptomycin. Cells were detached with trypsin-EDTA solution and passed for subculture when 90% confluency was reached. The synovial cells used were of passage 5.

Proliferating synovial fibroblasts from adjuvant arthritis were treated with phenylbutyrate or trichostatin A, and proliferation was determined by incorporation of ³H-thymidine, which was present during last 24 hours of culture. 4-phenylbutyrate (Merck) dissolved in sterile water with NaOH and adjusted to pH 7.2, or sodium phenylbutyrate (Triple Crown, U.S.A) dissolved in sterile water or trichostatin A (sigma) dissolved in ethanol were used. Nuclei from synovial fibroblasts were isolated by lysis of the cells in a buffer containing 10 mM Tris-HCl (pH 6.5), 50 mM sodium bisulfite, 1% Triton X-100, 10 mM MgCl₂, and 8.6% sucrose and use of a Dounce homogenizer. Histones were isolated by means of acid extraction. Isolated histones (5 μg) were then separated in 15% polyacrylamide-0.1% sodium dodecyl sulfate minigels and transferred to nitrocellular filters. Hyperacetylated histones were detected by use of the antibody that specifically recognizes the hyperacetylated form of histone H3 (upstate biotechnology).

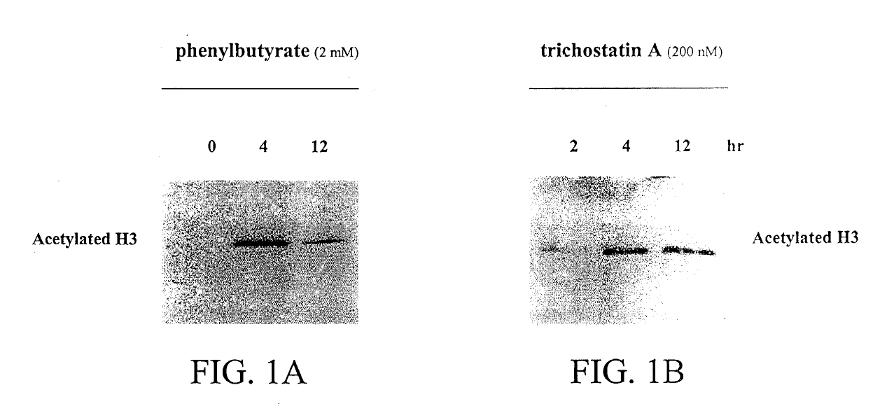

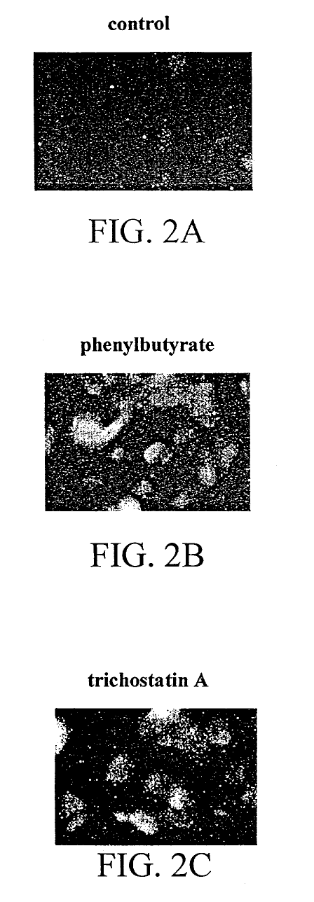

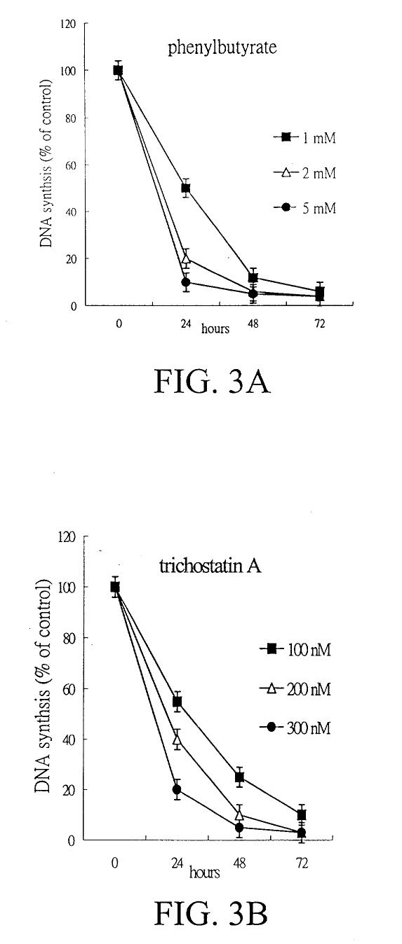

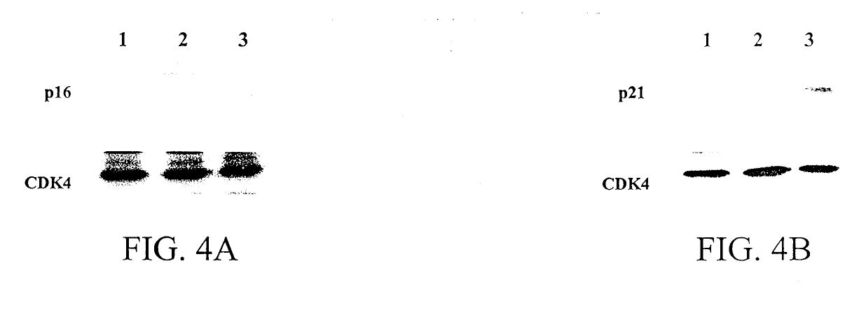

FIGS. 1A and 1B are western blot result showing that Phenylbutyrate and trichostatin A induce an accumulation of acetylated histones. FIGS. 2A-2C are photographs of immunofluorescence with anti-acetylated H3 antibody (upstate biotechnology). The synovial fibroblasts were treated with or without phenylbutyrate (2 mM) or trichostatin A (200 nM) for 6 h, and then stained with FITC-conjungated anti-acetylated H3 antibody. Phenylbutyrate and trichostatin A induce an accumulation of acetylated histones. FIG. 3A and 3B are diagrams showing that phenylbutyrate and trichostatin A exhibit a dose- and time- dependent growth inhibition in synovial fibroblasts from adjuvant arthritis. The results were expressed as percentage value relative to the control culture (no drug addition). Data presented are the mean±SE of triplicate determinations from two experiments. FIGS. 4A and 4B are western blot results showing that phenylbutyrate (2 mM) and trichostatin A (200 nM) treatment for 12 h induce upregulation of p16 and p21, cell cycle inhibitors, in synovial fibroblasts from adjuvant arthritis. Lane 1: control (no any treatment); lane 2: phenylbutyrate-treated; lane 3: trichostatin A-treated.

### EXAMPLE 2

### Various Topical Compositions-Oleaginous Ointment, Cream, and Gel.

A. Preparation of an Oleaginous Ointment of Phenylbutyrate:

470 g of white petrolatum (Riedel-de Haen), 25 g of paraffin wax 50/52 (local supplier), and 5 g of 4-phenylbutyrate (Merck) were mixed in a beaker and heated at 70° C. to form a paste. The paste was stirred at 400 rpm for 1 hour, and then cooled at room temperature.

B. Preparation of an Oleaginous Ointment of Phenylbutyrate:

65 g of white petrolatum (Riedel-de Haen), 15 g of cetyl alcohol (Riedel-de Haen), 260 g of soft paraffin (Merck), 155 g of liquid paraffin (Merck), and 5 g of 4-phenylbutyrate (Merck) were mixed in a beaker and heated at 70° C. to form a paste. The paste was stirred at 400 rpm for 1 hour, and then cooled at room temperature.

C. Preparation of Cream of Phenylbutyrate:

Part I: 70 g of Tefose 63®, 20 g of Superpolystate®, 10 g of Coster 5000®, 15 g of Myriyol 318®, 15 g of Coster 5088®, and 15 g of GMS SE® (all commercially available from local supplier) were mixed in a beaker and heated at 70° C.

Part II: 5.739 g of sodium 4-phenylbutyrate (Triple Crown America, Inc.), 0.125 g of methylparaben (Merck), 0.075 g of propylparaben (Merck), and 149.061 g of deionized water were mixed in a beaker and heated at 70° C.

The part II was slowly added into the part I and continually stirred at 400 rpm for 5 minutes to form a mixture. 2% Stabileze QM® (prepared by dissolving 2 g of Stabileze QM® in 98 g of deionized water, heating and stirring at 70° C. to form a paste, and cooling at room temperature) was added into the mixture and stirred for 5 minutes. The pH of the mixture was adjusted to 5.34 with 0.85% phosphoric acid (Merck), and stirred at 600 rpm for 20 minutes. The mixture was cooled at room temperature.

D. Preparation of Gel of Phenylbutyrate:

Part I: 10 g of Stabileze QM® and 232.035 g of deionized water were mixted in a beaker and heated at 70° C.

Part II: 5.739 g of sodium 4-phenylbutyrate (Triple Crown America, Inc.), 0.125 g of methylparaben (Merck), 0.075 g of propylparaben (Merck), 232.035 g of deionized water, and 20 g of 10%NaOH were mixed in a beaker and heated at 70° C.

The part II was slowly added into the part I and continually stirred with 400 rpm for 20 minutes to form a mixture. The mixture was cooled at room temperature.

E. Preparation of Gel of Phenylbutyrate:

Part I: 10 g of Stabileze QM® and 380.561 g of deionized water were mixed in a beaker and heated at 70° C.

Part II: 5.739 g of sodium 4-phenylbutyrate (Triple Crown America, Inc.), 0.125 g of methylparaben (Merck), 0.075 g of propylparaben (Merck), 83.5 g of 1,2-propandiol, and 20 g of 10%NaOH were mixed in a beaker and heated at 70° C.

The part II was slowly added into the part I and continually stirred at 400 rpm for 20 minutes to form a mixture. The mixture was cooled at room temperature.

F: Preparation of Sustained Release Formulations of Phenylbutyrate:

Two formulations were prepared according to the compositions listed in the Table 1.

TABLE 1
Compositions of two sustained release formulations

No. of formulation

| Composition | Tri-s-04 | Tri-s-05 |
|---|---|---|
| PF-127 ® (BASF Inc.)* | 2 | 4 |
| Sodium carboxy- | 12 | 12 |
| methylcellulose* | | |
| Deionized water | 82.8523 | 80.8523 |
| Sodium 4-phenylbutryate | 1.1477 | 1.1477 |
| 85% phosphoric acid | 2 | 2 |
| pH | 5.93 | 6.01 |

*PF-427 ® is the base of the compositions, and sodium carboxymethylcellulose is a thickening agent.

G: Preparation of Liposomal Formulation of Phenylbutyrate:

In this liposomal formulation, egg phosphatidylcholine (EPC) and cholesterol were used in equi- or different-molar concentrations as primary lipid components. Various liposomes located with 4-phenylbutyrate were obtained by varying the lipid:phenylbutyrate ratio. Liposomes were prepared by thin film hydration, sized by membrane extrusion, and physically evaluated.

H: Preparation of Ointment of Trichostatin A:

472.5 g of white petrolatum (Riedel-de Haen), 27 g of paraffin wax 50/52 (local supplier), and 0.5 g of trichostatin A (sigma) were mixed in a beaker and heated at 70° C. to form a paste. The paste was stirred at 400 rpm for 1 hour, and then cooled at room temperature.

I. Preparation of an Oleaginous Ointment of Trichostatin A:

67.5 g of white petrolatum (Riedel-de Haen), 16 g of cetyl alcohol (Riedel-de Haen), 260.5 g of soft paraffin (Merck), 155.5 g of liquid paraffin (Merck), and 0.5 g of trichostatin A (sigma) were mixed in a beaker and heated at 70° C. to form a paste. The paste was stirred at 400 rpm for 1 hour, and then cooled at room temperature.

### Example 3

### Effects of Phenylbutyrate and Trichostatin A on Suppression of Inflammation of Skin, Joints and Soft Tissues In Connective Tissue Disease by Topical Administration.

The animal model for connective tissue disease was established according to the report by Winter C A, et al., Arthritis Rheum. 1966, 9:394-404. Groups of 5 Long Evans rats weighing 150±20 g were used. The 10% of phenylbutyrate cream and 1% of trichostatin A ointment at a dose of 200 mg/paw and 10 mg/paw, respectively, were applied topically twice daily for 18 consecutive days. A well-ground suspension of killed Mycobacterium tuberculosis (DIFCO, USA; 0.3 mg in 0.1 ml of light mineral oil; Complete Freund's Adjuvant, CFA) was administered into the subplantar region of the right hind paw immediately after first dosing on first day (denoted day 1). Hind paw volume was measured by Plethsmometer (Cat. No. 7150, UGO BASILE, Italy) and Water cell (25 mm diameter, Cat. No. 7157, UGO BASILE, Italy) on day 0 (before CFA treated), 1, 5, 10, and 15 after CFA of right paw (with CFA), and day 0, 14, and 18 of left paw (without CFA); rats were weighed just before first dose and 1 hour after final dose. Anti-inflammatory activity in this model was denoted by values calculated during the Acute Phase (the right hind paw volume) as well as the Delayed Phase (the contralateral left hind paw volume). For CFA-injected vehicle control rats, animals were also weighed on day 0 and day 18; CFA-injected vehicle control animals generally gain between 20 to 40 g body weight over this time period. A 30% or more reduction in paw volume relative to vehicle treated controls was considered significant.

Table 2 shows the results which indicate that the 10% of phenylbutyrate cream and the 1% of trichostatin A ointment both have local anti-inflammatory effects on skin, joints, and soft tissues in connective tissue disease with prevention or amelioration of joint destruction, skin ulcer, soft tissue swelling, fibrosis and necrosis, and preservation of limb function.

TABLE 2
Anti-inflammatory activity.
(A). In Acute Phase (unilateral ankle joint)

| | | | % Inhibition relative to vehicle | | | |
|---|---|---|---|---|---|---|
| | | | treated | | | |
| | | | Day | Day | Day | Day |
| Compound | Route | Dose | (1-0) | (5-0) | (10-0) | (15-0) |
| 10% of Topic | Top- | 200 mg/paw × | 16 | 18 | (40) | (31) |
| phenylbutyrate | ical | 2 × 18 | | | | |
| cream | | | | | | |
| 1% of | Top- | 200 mg/paw × | −5 | 1 | 6 | −5 |
| phenylbutyrate | ical | 2 × 18 | | | | |
| cream | | | | | | |
| Cream | Top- | 200 mg/paw × | — | — | — | — |
| vehicle | ical | 2 × 18 | | | | |
| 1% of | Top- | 10 mg/paw × | 22 | 29 | (50) | (41) |
| trichostatin | ical | 2 × 18 | | | | |
| A ointment | | | | | | |
| Ointment | Top- | 10 mg/paw × | — | — | — | — |
| vehicle | ical | 2 × 18 | | | | |
| 0.5% CMC* | PO | 10 mg/kg × 5 | — | — | — | — |
| Hydrocortis: | PO | 30 mg/kg × 5 | (35) | (35) | (39) | 23 |
| one | | | | | | |

(B). In Delayed Phase (contralateral ankle joint)

| | | | % Inhibition relative to | | |
|---|---|---|---|---|---|
| | | | vehicle treated | | |
| Compound | Route | Dose | Day (14-0) | Day (18-0) | Day (18-14) |
| 10% of | Topical | 200 mg/paw × | 6 | 4 | 0 |
| phenylbuty | | 2 × 18 | | | |
| rate cream | | | | | |
| 1% of | topical | 200 mg/paw × | 6 | 4 | 0 |
| phenylbuty | | 2 × 18 | | | |
| rate cream | | | | | |
| Cream vehicle | topical | 200 mg/paw × | — | — | — |
| | | 2 × 18 | | | |
| 1% of | topical | 10 mg/paw × | 7 | 6 | 0 |
| trichostatin | | 2 × 18 | | | |
| A ointment | | | | | |
| Ointment vehicle | topical | 10 mg/paw × | — | — | — |
| | | 2 × 18 | | | |
| 0.5% CMC* | PO | 10 mg/kg × 5 | — | — | — |
| Hydrocortisone | PO | 30 mg/kg × 5 | (30) | 27 | 20 |

*0.5% carboxymethylcellulose and hydrocortisone are commercially available from Sigma, USA.

Referring to Table 2, systemic, orally administered hydrocortisone is used for clinically treating connective tissue disease in acute stages. A marked inhibition effect of hydrocortisone is shown on day 1; however, the effect reduces after day 10. 0.5% Carboxymethylcellulose is the excipient of hydrocortisone, and used herein as a control group. On the other hand, the initial effects of the 10% phenylbutyrate cream and 1% trichostatin A ointment are not obvious, but 40% and 50% of inhibition are observed since day 10, and even 31% and 41% of inhibition are maintained on day 15, respectively. There is a dose response relationship in phenylbutyrate since the 1% of phenylbutyrate cream is not as effective as the 10% cream.

Referring to Table 2, the systematic inhibition effect of hydrocortisone is partially shown in delayed phase (i.e. left paw), while that of the phenylbutyrate and trichostatin A are not significant. The results indicate that the topical formulations of the invention have local rather than systematic efficacy, which is useful in the topical application to external regions.

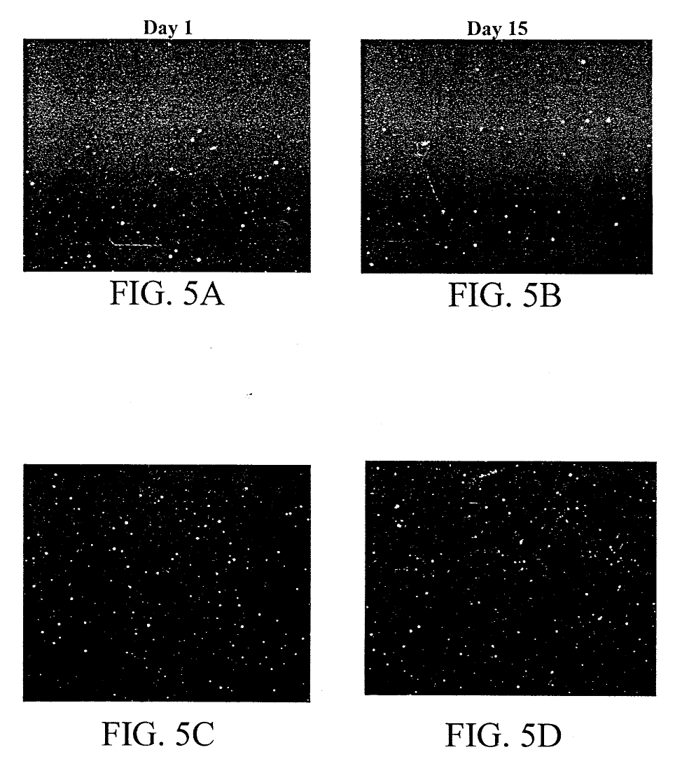

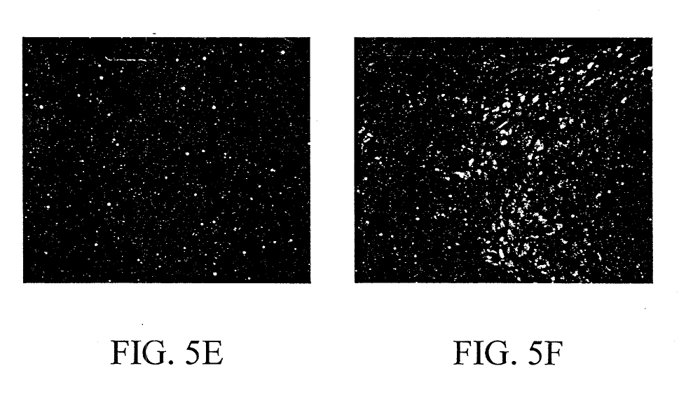

FIGS. 5A-5F are photographs showing treatment with 10% of phenylbutyrate cream or 1% trichostatin ointment induces accumulation of acetylated histones in vivo. On Day 1 and Day 15, the soft tissues from connective tissue disease treated with phenylbutyrate or trichostatin A are shown to have increased acetylated H3 by immunofluorescence (Santa Cruz).

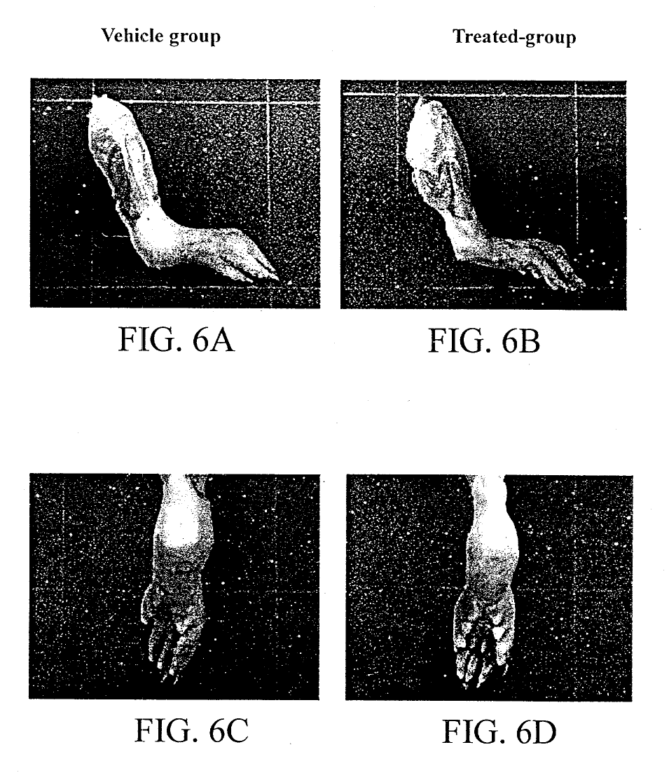

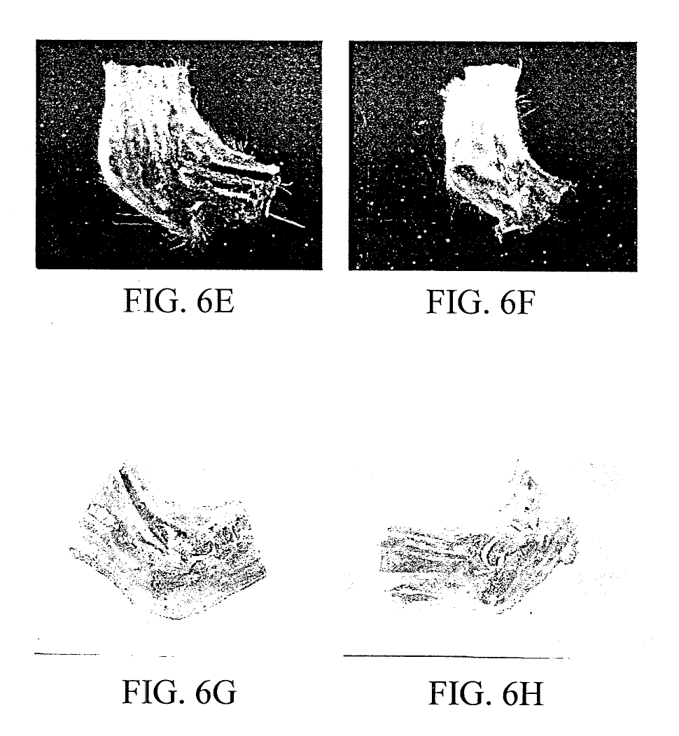

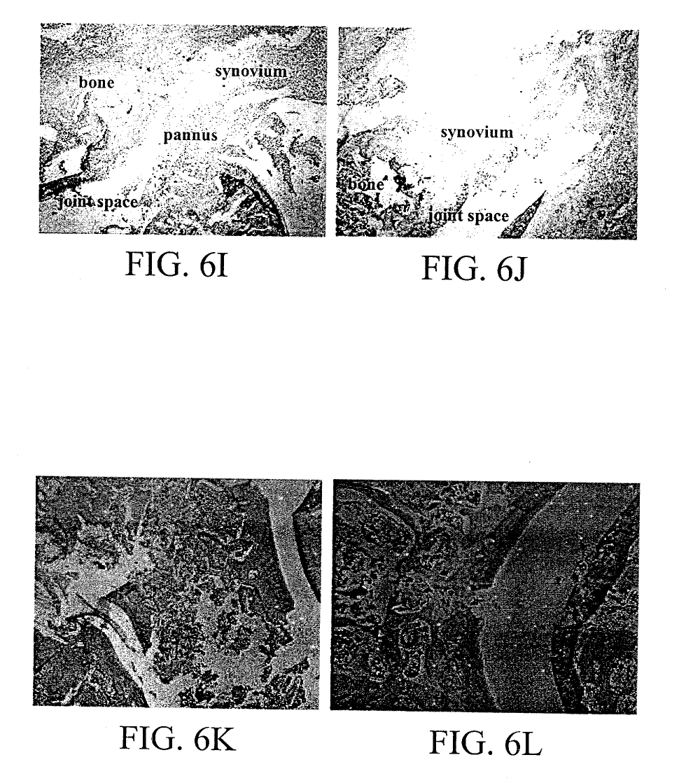

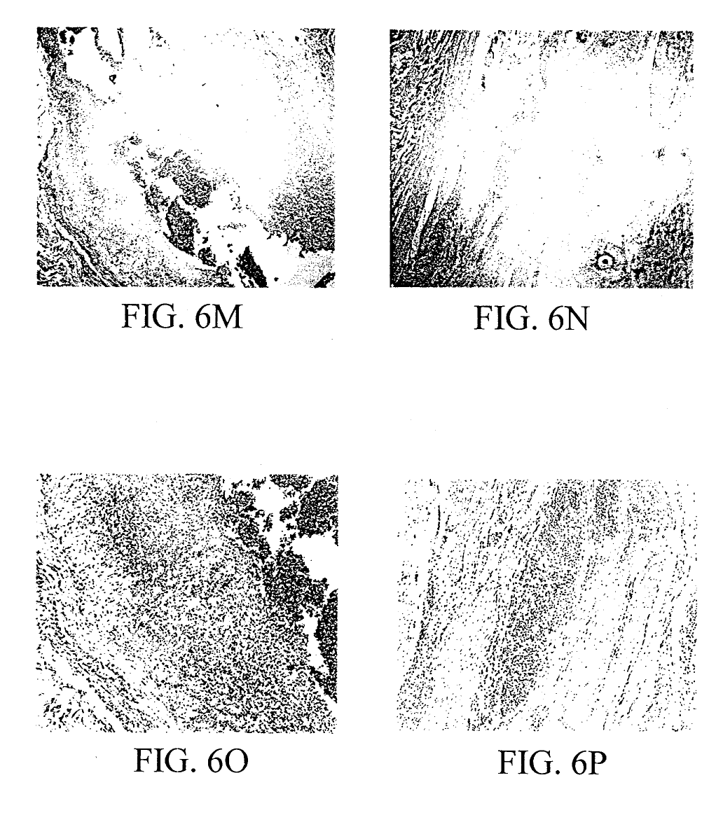

FIGS. 6A-6P are photographs showing that phenylbutyrate and trichostatin A have similar effects on suppression of the connective tissue disease. The left panel is the vehicle group, and the right panel is the treated group. FIGS. 6A and 6B are lateral views of ankle and plantar joints. Comparison of the lateral views shows suppression of the swelling of ankle and plantar joints in the treated group. FIGS. 6C and 6D are back views of ankle and plantar joint. Healing is promoted in the plantar skin wound in the treated group. This skin wound resulted from killed Mycobacterium tuberculosis injected into the sub-plantar region to produce connective tissue disease. FIGS. 6E and 6F are cross-sections (sagittal cut). The joint structure in the paw is swollen and deteriorated in the vehicle group but preserved in the treated group. FIGS. 6G and 6H are micro-sections confirming that panus formation is prevented, soft tissue swelling is decreased, and the joints are preserved in the treated group. FIGS. 6I and 6J are Histology (40×) showing severe synovial hyperplasia, pannus formation and joint destruction in the vehicle group but only moderately synovial hyperplasia is noted and no pannus is formed in the treated group. FIGS. 6K and 6L are higher magnification (100×) to demonstrate that the cartilage and bone are deteriorated in the connective tissue disease but preserved after treatment with histone deacetylase inhibitors. FIGS. 6M and 6N are histology (40×) and the vehicle group shows severe dermal swelling, subcutaneous thick fibrosis with microabscess formation and diffuse lymphocyte infiltration, but the treated group shows normal dermal structure with only small focal lymphocyte aggregation without swelling. FIGS. 6O and 6P are higher magnification (100×). The vehicle group shows diffuse lymphocyte infiltration and fibrosis in subcutaneous tissue. The treated group only shows focal lymphocyte aggregation, and the muscle bundles and connective tissues are still distinguishable.

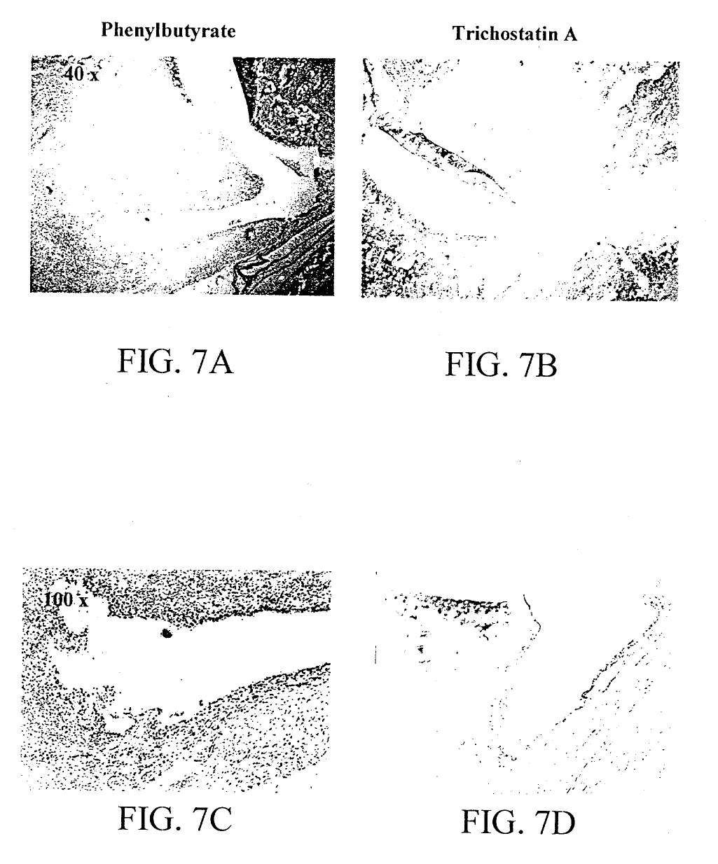

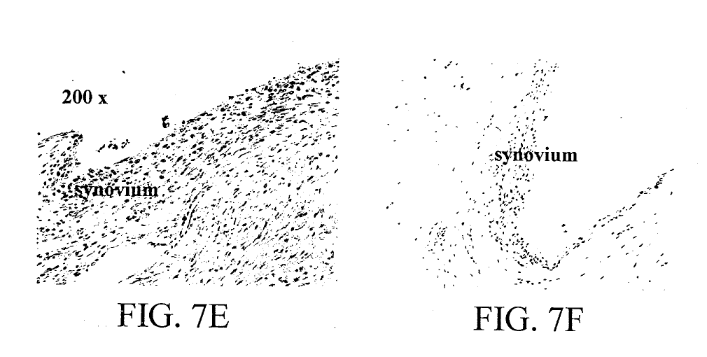

As shown in Table 2 and FIGS. 7A-7F, trichostatin A with 1% ointment is more potent than phenylbutyrate with 10% cream. The lymphocyte infiltration, soft tissue swelling and synovial hyperplasia are suppressed more effectively in the 1% trichostatin A ointment treated group than in the 10% phenylbutyrate cream treated group.

### EXAMPLE 4

### Effect of Phenylbutyrate Inhibitors On the Cell Cycle Regulator In the Inflamed Invasive Synovium of Connective Tissue Disease.

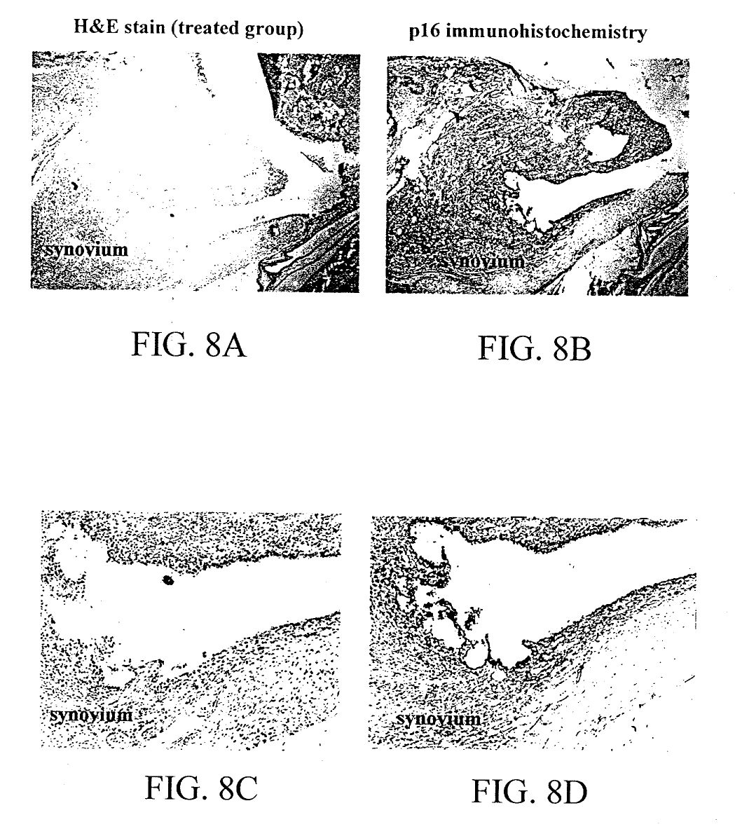

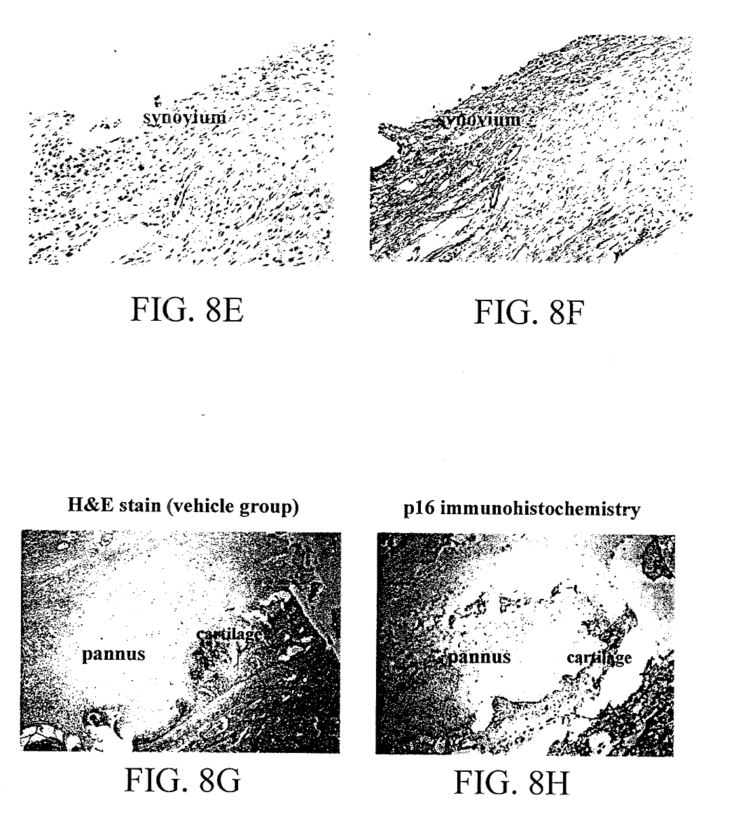

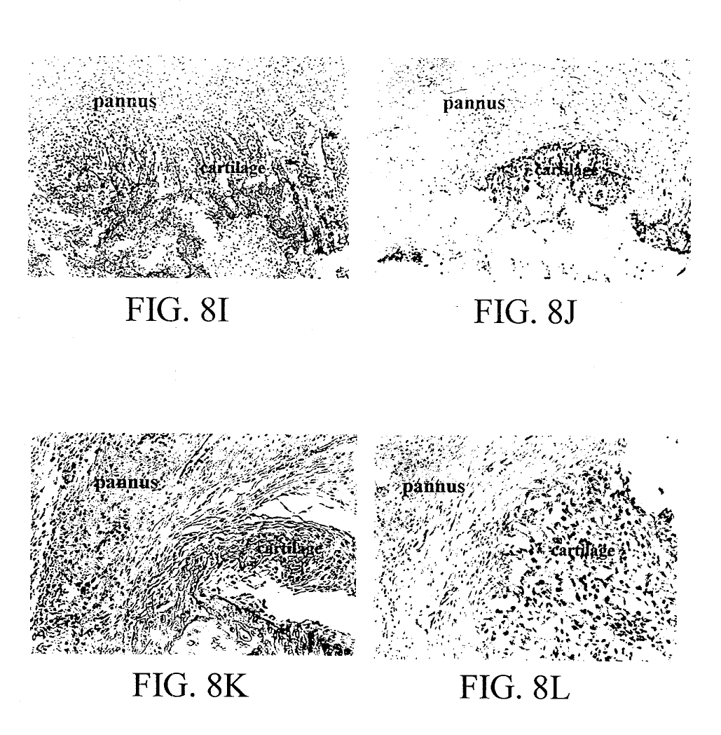

FIGS. 8A-8F are from lower magnification to higher magnification showing the results of H&E stain (left panel), and immunohistochemistry (right panel) which was performed to demonstrate that the cell cycle inhibitor, p16, is upregulated in the synovium of connective tissue disease, and pannus formation is blocked after the phenylbutyrate treatment although there is still some lymphocyte infiltration in the synovium.

FIGS. 8G-5L are from lower magnification to higher magnification showing the severe synovial hyperplasia and panus formation in connective tissue disease deteriorating the cartilage and bone of joints (left panel: H&E stain). In contrast, there is no p16 staining in the invasive synovium of the vehicle group when compared to the synovium in the treated group (right panel: immunohistochemistry).

The results demonstrate that phenylbutyrate can induce cell cycle arrest in the synovial fibroblasts of connective tissue disease to prevent pannus formation and joint destruction.

### EXAMPLE 5

### Effect of Phenylbutyrate On the Proinflammatory Mediator In the Connective Tissues of Connective Tissue Diseases.

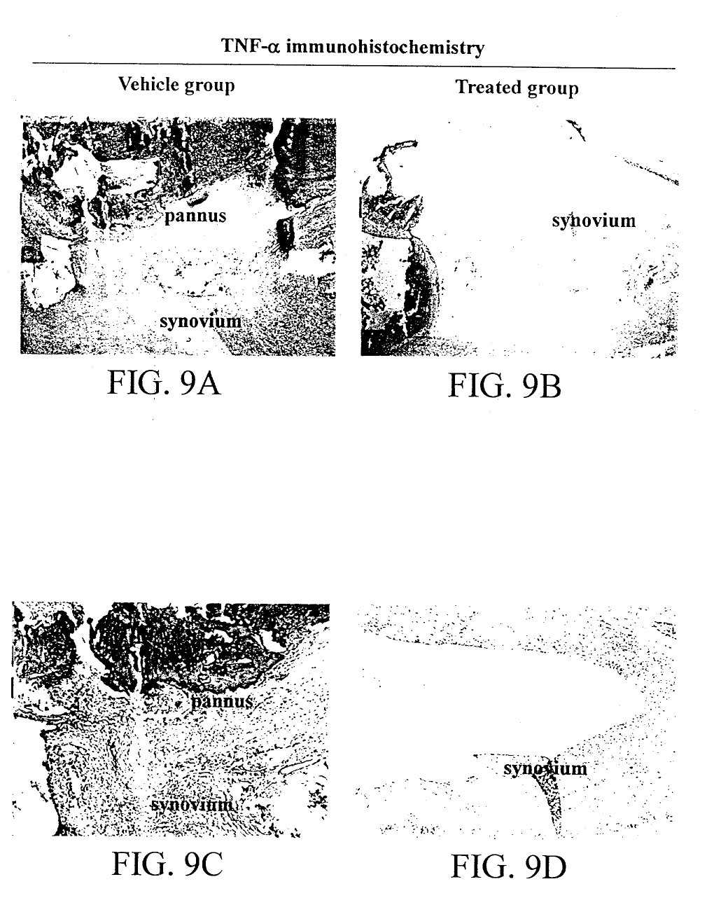

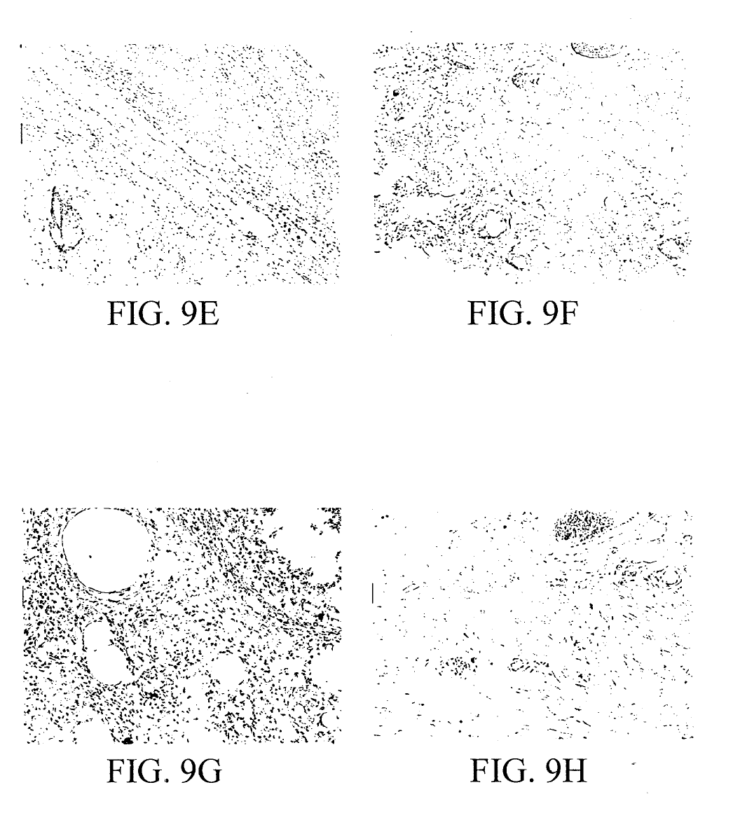

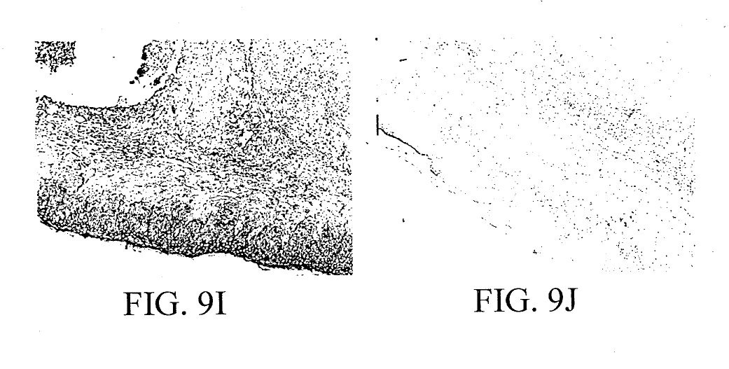

FIGS. 9A-9J show that TNF-A is widespread in the connective tissue disease with inflammation of skin, subcutaneous tissues and joint in the vehicle group but only small amount of TNF-α was stained in the skin in the phenylbutyrate-treated group. This result indicates that phenylbutyrate can suppress the proinflammatory mediator, TNF-α, in the connective tissues (including synovium lining the joint, subcutaneous tissue, dermis, and epidermis) of connective tissue disease.

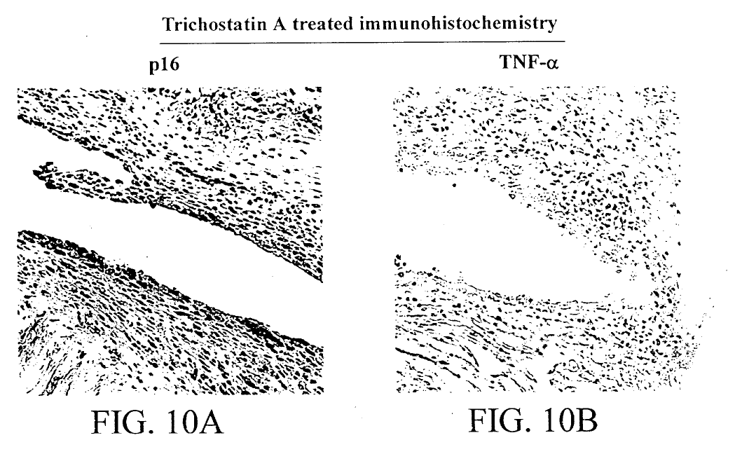

Moreover, trichostatin A also has the same effects on upregulation of p16 and downregulation of TNF-α as phenylbutyrate. As shown in FIG. 10A, trichostatin A upregulates p16 expression in the thin synovium lining the joint space. In contrast, there is no TNF-α staining in soft tissues in the connective tissue disease after trichostatin A treatment (10B).

Referring to Examples 4 and 5, phenylbutyrate and trichostain A simultaneously coordinate upregulation of a cell cycle inhibitor, p16, and downregulation of an inflammatory mediator, TNF-α, in connective tissue disease. This result suggests that histone deacetylase inhibitors are effective for the treatment of connective tissue diseases of which cell cycle has dysregulation, and proinflammatory cytokine aberrant response are characteristics.

The results in the present invention indicate that histone deacetylase inhibitors provide a novel therapeutic potential in the treatment of connective tissue diseases consisting of rheumatoid arthritis, systemic lupus erythematosus, progressive systemic sclerosis, sjogren's syndrome, dermatomyositis, and mixed connective tissue disease with inflammation of skin, joints, and soft tissues due to altered patterns of immunoregulation as autoimmune disorders.

In conclusion, at least two unrelated histone deacetylase inhibitors are active compounds for the treatment of connective tissue diseases. The present invention also relates to a method for the treatment of humans or animals afflicted with connective tissue diseases, comprising administering to the subject an effective amount of a histone deacetylase inhibitor in particular trichostatin A and phenylbutyrate or a pharmaceutically acceptable salt thereof and optionally a suitable excipent.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally and functionally analogous to histone deacetylase inhibitors described above can also be used to practice the present invention. Thus, other embodiments are also within the claims.

What is claimed is:

1. A pharmaceutical composition for the treatment of connective tissue disease, comprising:
a histone deacetylase inhibitor and
a pharmaceutically acceptable carrier or a pharmaceutically acceptable salt thereof.

2. The pharmaceutical composition as claimed in claim 1, wherein the connective tissue disease comprises rheumatoid arthritis, systemic lupus erythematosus, progressive systemic sclerosis, sjogren's syndrome, dermatomyositis, or mixed connective tissue disease.

3. The pharmaceutical composition as claimed in claim 1, wherein the histone deacetylase inhibitor comprises valproic acid, phenylbutyrate, depudecin, trapoxin A, depsipeptide, trichostatin A, oxamflatin, or benzamide.

4. A method for treating connective tissue disease, comprising administration to humans or animals in need of an anti-inflammatory treatment a therapeutically effective amount of a pharmaceutical composition comprising a histone deacetylase inhibitor and a pharmaceutically acceptable carrier or a pharmaceutically acceptable salt thereof.

5. The method as claimed in claim 4, wherein the histone deacetylase inhibitor is present in an amount from 0.1 to 50% by weight of the compositions.

6. The method as claimed in claim 4, wherein the pharmaceutical composition is administered non-orally.

7. The method as claimed in claim 6, wherein the composition is a cream, an ointment, a gel, a lotion, a patch, a suppository, a liposome formation, an injection solution, or a drip infusion.

8. The method as claimed in claim 4, wherein the histone deacetylase inhibitor comprises trichostatin A or a pharmaceutically acceptable salt thereof.

9. The method as claimed in claim 4, wherein the histone deacetylase inhibitor comprises phenylbutyrate or a pharmaceutically acceptable salt thereof.

10. The method as claimed in claim 4, wherein the connective tissue disease comprises rheumatoid arthritis.

11. The method as claimed in claim 4, wherein the connective tissue disease comprises systemic lupus erythematosus.

12. The method as claimed in claim 4, wherein the connective tissue disease comprises progressive systemic sclerosis.

13. The method as claimed in claim 4, wherein the connective tissue disease comprises sjogren's syndrome.

14. The method as claimed in claim 4, wherein the connective tissue disease comprises dermatomyositis.

15. The method as claimed in claim 4, wherein the connective tissue disease comprises mixed connective tissue disease.
